(19) United States
(12) Patent Application Publication
(10) Pub. No.: US 2009/0198184 A1
(43) Pub. Date: Aug. 6, 2009

(54) Percutaneous biomedical devices with regenerative materials interface

(76) Inventors: David C. Martin, Ann Arobor, MI (US); Antonio Peramo, Ann Arbor, MI (US); Steven A. Goldstein, Ann Arbor, MI (US)

Correspondence Address: HARNESS, DICKEY & PIERCE, P.L.C., P.O. BOX 828, BLOOMFIELD HILLS, MI, 48303, US

(21) Appl. No.: 12/322,716

(22) Filed: Feb. 5, 2009

Related U.S. Application Data
(60) Provisional application No. 61/063,704, filed on Feb. 5, 2008.

(51) Int. Cl. A61M 5/00; A61M 1/00; A61M 25/14; A61B 17/60; A61B 17/58; A61B 17/70; A61F 2/82; A61F 2/18; A61F 2/60; A61F 2/54

(52) U.S. Cl. 604/151; 604/288.04; 604/523; 604/264; 606/54; 606/53; 606/246; 623/1.15; 623/10; 623/27; 623/57

(57) ABSTRACT

A percutaneous biomedical device comprising a body having a lumen extending longitudinally at least partially through the body, an implantable interface region disposed on the body, the implantable interface region having a plurality of radially extending conduits through the body, each of the conduits are in fluid communication with the lumen and in fluid communication with an exit port. The exit ports extrude a skin-interface composition between the subject's skin and the percutaneous biomedical device. Methods for implanting a percutaneous biomedical device includes implanting a percutaneous biomedical device percutaneously in a subject and aligning the exit ports of the implantable interface region between the epidermis and the hypodermis skin layers of the subject.

### CROSS-REFERENCE TO RELATED APPLICATIONS

This application claims the benefit of U.S. Provisional Application No. 61/063,704, filed on Feb. 5, 2008. The entire disclosure of the above application is incorporated herein by reference.

### GOVERNMENT RIGHTS

This invention was made with government support under grant W911NF-06-1-0218 awarded by the Army Research Office. The government has certain rights in the invention.

### FIELD

The present technology relates to implantable percutaneous biomedical devices having a biocompatible implantable interface region and methods of implanting percutaneous biomedical devices to prevent infection and inflammation of the implant-skin interface.

### BACKGROUND

The statements in this section merely provide background information related to the present technology and may not constitute prior art. In the past decade, there have been numerous advances in the development of transcutaneous devices including nails, fixator pins, screws, catheters, glucose sensors, prostheses and osteointegrative prosthetic limb devices for amputees to name but a few. The skin of a patient who wears a transcutaneous device is subject to numerous abuses.

Typically, the implantable percutaneous biomedical devices are made of stainless steel, titanium or polymeric materials. The soft tissue area in contact with the implantable device is the one that most likely will present an inflammatory response, in particular, in the dermal and epidermal layers of the skin. In addition, the interface remains “unsealed” enabling the seeding of microbial based infections. The state of the skin is of utmost importance, for example, to an amputees' ability to use a prosthesis particularly an osseointegrated prosthesis limb device. If the normal skin condition cannot be maintained despite daily wear and tear, the prosthesis cannot be worn, no matter how accurate the integrated limb device may be.

Poor hygiene may be an important factor in producing some pathologic conditions of the transcutaneous-skin interface. If a routine cleansing program is not employed, bacterial and fungal infections, nonspecific eczematization, intertrigo, and persistence of infected epidermoid cysts can eventuate.

Bacterial folliculitis and furuncles or boils are often encountered in amputees with hairy, oily skin, with the condition aggravated by sweating and rub from the transcutaneous device. It is usually worse in the late spring and summer when increased warmth and moisture from perspiration promote maceration of the skin in contact with the transcutaneous device. Ordinarily this process is not serious, but sometimes, especially in diabetics, it can progress to furuncles, cellulitis, or an eczematous weeping, crusted, superficial, impetiginized pyoderma. In some patients, therapy may require a wet compress, incision and drainage of the infectious interface after localization and oral or parenteral use of antibiotics, and local application of bacteriostatic or bactericidal agents.

In order to reduce or possibly eliminate the foreign-body-like reaction and microbial infection, the present technology provides for a biocompatible material at the skin-device interface that works as a tissue-integrative device.

Several fully internal osseointegrated devices have been described for orthopedic applications. In some cases, titanium joint arthroplasties incorporating osseointegration principles have been implemented and have gained usage worldwide. Notwithstanding the mechanical advantages of these transcutaneous devices however, concerns have been raised regarding their transcutaneous effects. The concerns are not about the integration of the device with bone but about the development of pathways around the implant through soft tissues, where environmental contamination have caused titanium corrosion and bone infection. Corrosion and infection could lead to further loss of bone length, resulting in a shorter residual limb and decreased function. Infection, bone loss, and loosening are common in transcutaneous implants.

Many responses to the problems associated with transcutaneous devices have focused on eliminating contact between the bone and the environment and restricting contamination of the prosthesis and bone. To this effect, the field has generally adopted strategies that encourage dermal and epithelial growth into prosthetic surfaces. However, problems associated with such biointegration stem from the fact that the transcutaneous devices may not remain completely stationary, i.e. these devices are dynamic, they can move under physical stresses imposed externally or internally. As a general principle, the human body reacts to insoluble foreign bodies placed within it either by extruding them (if they can be moved and an external wall is close at hand) or by walling them off by exactly the same process as wound granuloma formation. In other cases the local host response to an implant in contact with epithelial tissue will be the formation of a pocket or pouch continuous with the adjacent epithelial membrane, a process called “marsupialization” due to the structural similarity to a kangaroo's pouch. In the case of the external epithelium i.e. skin, marsupialization results in the extrusion of the implant from the host unless the implant is anchored in the deep connective tissue or other deep tissue.

The present technology provides for implantable devices that can remain partially implanted transcutaneously for an extended period of time without the deleterious effects described above.

### SUMMARY

The present technology relates to highly biocompatible percutaneous biomedical devices that can be partially implanted percutaneously into a patient. In particular, the present technology provides a body having a lumen extending longitudinally at least partially through the body, an implantable interface region disposed along the body. The implantable interface region has a plurality of radially extending conduits through the body, each of the conduits in fluid communication with the lumen and in fluid communication with an exit port. The implantable interface region is placed between the stratum corneum and the hypodermis layers of the subject's skin. The implantable interface region can include a plurality of radially extending conduits, each of the conduits is in fluid communication with a lumen and/or a reservoir and in fluid communication with an exit port to extrude a skin-interface composition between the subject's skin and the percutaneous biomedical device.

The lumen can store a skin-interface composition that is extruded at the skin-layer device interface. The radially extending conduits are each in fluid communication with the lumen and an exit port.

In another aspect the implantable interface region includes a first diameter and a second diameter, said second diameter being smaller than said first diameter and said second diameter defining a circumferential depression with a plurality of exit ports.

In another aspect, the present technology provides for methods of implanting a transcutaneous device. The method includes the steps of: providing a percutaneous biomedical device having an implantable interface region and a lumen for receiving a fluid comprising a skin-interface composition. The implantable interface region includes a plurality of radially extending conduits, such that the conduits are in fluid communication with the lumen and each conduit is in fluid communication with an exit ports; implanting the percutaneous biomedical device across the skin and in communication with a tissue and aligning said plurality of exit ports adjacent to a skin layer. Once implanted, the percutaneous biomedical device can form a skin layer interface by extruding a biocompatible, protective and nutritive skin-interface composition through the exit ports to contact the one or more skin layers.

Further areas of applicability will become apparent from the description provided herein. It should be understood that the description and specific examples are intended for purposes of illustration only and are not intended to limit the scope of the present technology.

### DRAWINGS

The drawings described herein are for illustration purposes only and are not intended to limit the scope of the present technology in any way.

FIG. 1A is a side elevation view of a percutaneous biomedical device which includes an implantable interface region in the form of a prosthesis frame in accordance with the present technology.

FIG. 1B is a side elevation view of a percutaneous biomedical device which includes an implantable interface region having a circumferential depression, the exit ports are disposed in the circumferential depression in accordance with the present technology.

FIG. 2 is a side view of a cylindrical percutaneous biomedical device having a circumferential depression having a rectangular cross-section, the circumferential depression having a smaller diameter than the implantable device member. A plan cross-section view of the cylindrical percutaneous biomedical device taken through a plane dissecting the circumferential depression is shown illustrating the orientation of the conduits and the lumen in relation to the exit ports. The cylindrical percutaneous biomedical device is shown with a mesh around the exit ports in accordance with the present technology.

FIG. 3 is an illustration of a cylindrical percutaneous biomedical device connected to an external reservoir in accordance with the present disclosure.

FIG. 4 is an illustration showing an embodiment of the implantable interface region and a central lumen in fluid communication with four exit ports in phantom. The implantable interface region has an internal reservoir connected to an external reservoir. The flow of the skin-interface composition from the external reservoir to the exit ports is controlled by an external pump and flow meter in accordance with the present technology.

FIG. 5 is an illustration of a cut away section of a percutaneous biomedical device implanted percutaneously depicting the implantable interface region. The implantable interface region has an internal reservoir in fluid communication with a pump that delivers the skin-interface composition from the internal reservoir to the lumen and then out via the conduits connected to an exit port thereby forming a skin layer interface at the junction between at least a partial area of the implantable interface region and one or more skin layers in accordance with the present technology.

FIG. 6 is a graphical representation of various skin layers consisting of the dermis and epidermis skin layers and includes an implantable interface region integrated with bone, the implantable interface region extruding a skin-interface composition at the skin layer interface providing a biocompatible matrix for the rejuvenation of skin cells and a natural barrier for the entry of infectious agents in accordance with the present technology.

FIG. 7 is an illustration of a prosthesis frame for supporting a limb prosthesis which includes an implantable percutaneous biomedical device member in the form of a prosthesis frame extruding a skin-interface composition from a plurality of exit ports in the implantable interface region of the biomedical device to the skin layers of the limb in accordance with the present technology.

FIG. 8 panels 1-3 depict microscopy images of haematoxylin and eosin (H&E) staining of skin explants percutaneously inserted with pins. Panel 1 represent microscopy images of a representative skin implant taken after implantation of pins and no skin-interface composition extrusion from the pin. Panel 2 represent microscopy images of a representative skin implant taken after implantation of pins extruding saline through the exit ports. Panel 3 represent microscopy images of a representative skin implant taken after implantation of pins extruding a skin-interface composition containing hyaluronic acid and dermatan sulfate (HA+DS).

### DETAILED DESCRIPTION

The following description is merely exemplary in nature and is not intended to limit the present technology, application, or uses.

The terminology used herein is for the purpose of describing particular example embodiments only and is not intended to be limiting. As used herein, the singular forms “a”, “an” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on”, “engaged To”, “connected to” or “coupled to” another element or layer, it may be directly on, engaged, connected or coupled to the other element or layer, or intervening elements or layers may be present. In contrast, when an element is referred to as being “directly on,” “directly engaged to”, “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath”, “below”, “lower”, “above”, “upper” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The present technology relates to percutaneous biomedical devices providing an enhanced biocompatible skin-device interface, herein referred to as a skin layer interpace. A percutaneous biomedical device comprising a body having a lumen extending longitudinally at least partially through the body, an implantable interface region disposed along the body, the implantable interface region having a plurality of radially extending conduits through the body, each of said conduits in fluid communication with the lumen and in fluid communication with an exit port. As used herein, the implantable interface region can be constructed to serve as the percutaneous biomedical device or the implantable interface region can be integrated with or is part of a percutaneous biomedical device. The implantable interface region can be a part (or in its entirety) of any implant that is percutaneously implanted across a Subject's skin layer for an extended period of time. As used herein, a percutaneous biomedical device contemplates all transcutaneous devices that are implanted into a subject for a period of time longer than 2 weeks and in which a portion of the transcutaneous device extends through the skin and exits to the outside of the subject. The percutaneous biomedical device can include orthopedic devices including: fixator pins, orthopedic or craniofacial pins, rods, screws; prosthetic limb frames; prosthetic osseointegrated devices; catheters; stents; leads; wires and flexible or inflexible tubular devices that remain partially inserted in a subject for an extended period of time, typically greater than 2-3 weeks.

The percutaneous biomedical device can optionally also include a reservoir, a pumping mechanism to pump the contents of the reservoir into the lumen or exit ports of the implantable interface region and a skin-interface composition. The optional components, including the reservoir and pumping mechanism can be placed within the percutaneous biomedical device or placed outside of the percutaneous biomedical device. Additionally, the reservoir and pumping mechanism can be coupled to the implantable interface region or percutaneous biomedical device though connectors, which enable the reservoir and pumping mechanism to be placed outside of the subject's body.

### Implantable Interface Region

The present technology provides a percutaneous biomedical device which allows the integration of the device with one or more layers of the skin. The percutaneous biomedical device can be any device that spans the epidermal, dermal and hypodermal skin layers of the subject. In some embodiments, the percutaneous biomedical device typically has an in vivo portion and an ex-vivo portion. The transcutaneous nature of the biomedical device is merely used to reference the in vivo portion of the transcutaneous being passed through the skin layers and placed within the body (in vivo). The portion of the percutaneous biomedical device that spans at least a portion of the epidermis and dermis layers can be referred to as the implantable interface region. Once the percutaneous biomedical device has been implanted, a skin-interface composition can be extruded from the implantable interface region's exit ports located between the stratum corneum of the epidermis and hypodermis layer of the subject's skin. The skin-interface composition is positively applied along at least a portion of the exterior surface of the implantable interface region in proximate contact with the one or more layers of the epidermal and dermal skin facilitating biological integration of the percutaneous biomedical device with the subject's skin. As used herein, the subject's skin comprises generally of three layers the epidermis layer, the dermis layer and the inner hypodermis layer that is composed largely of connective tissue. The movement of fluid from the hypodermis layer towards the epidermis is said to be superficial whereas, movement of fluid from the epidermis towards the hypodermis is said to be subcutaneously.

The implantable interface region 10 illustratively shown in FIGS. 1A and 1B are shown to have a generally cylindrical configuration. While shown as a cylinder, and having a circular cross-sectional shape, other shapes and cross-sectional shapes are contemplated, including square, rectangular, triangular, oval and other geometric shapes provided they can be positioned and situated at least partially between the subject's epidermis and hypodermis. The implantable interface region can also be a part of a percutaneous biomedical device, and can have a configuration that is compatible with the percutaneous biomedical device.

As illustrated in FIG. 1A, the implantable interface region 10 has an in vivo portion 12 and an ex-vivo portion 14. The in vivo portion 12 is illustrated to exemplify that the in vivo portion 12 can be in contact with a tissue (in vivo) within the subject, for example, a bone surface for integration with the percutaneous biomedical device or it can be a blood vessel, organ tissue, or other connective tissue or internal organ or tissue within the body. In some embodiments, the in vivo portion 12 can be part of the percutaneous biomedical device inserted into a lumen such as a catheter, stent or tube inserted within the body of a subject.

The implantable interface region 10 further includes an ex vivo portion 14. Ex vivo portion 14 can be the portion of the transcutaneous device that is situated outside (exterior to) the subject's skin. In some embodiments, ex vivo portion 14 can be connected to a prosthesis. Ex vivo portion 14 can also refer to the proximal portion of the percutaneous biomedical device in reference to where the device is being manipulated by the operator. Implantable interface region 10 can be part of a larger or longer percutaneous biomedical device, for example a drug delivery tube, stent or catheter.

The percutaneous biomedical device has an implantable interface region 10, which at least partially spans the skin between the stratum corneum and the hypodermis. A first skin interface region 16 is illustrated in FIGS. 1A and 1B in a canonical form, wherein the first skin interface region 16 spans at least, the skin layers between the stratum corneum and the hypodermis. The length of the first skin interface region 16 can vary widely, depending on the configuration of the percutaneous biomedical device and the width of the skin layers into which the implantable interface region 10 will be inserted. In some embodiments, the in vivo portion 12 can be inserted into bone, and the exit ports 30 can be placed in the epidermis and/or dermis and/or hypodermis layers of the skin. In some embodiments, the length of the first skin interface region 16 can range from about 3 mm to about 100 mm. Advantageously, the first implantable interface region 10 can be integrated into a percutaneous biomedical device having any dimensions necessary to fulfill its biomedical purpose.

As shown in FIGS. 1A and 1B, the implantable interface region has a plurality (more than one) exit ports 30 that can be spaced around the diameter of the implantable interface region 10. As shown in FIGS. 1A and 1B, the exit ports 30 are equidistantly spaced, but this is not absolutely necessary. The exit ports 30 can range in number and be spaced sufficiently apart to ensure coverage of the skin-interface composition substantially around the diameter of the implantable interface region 10. As noted above, the percutaneous biomedical device can integrate the implantable interface region 10 and therefore can have the same diameter as the implantable interface region 10. The exit ports 30 are illustrated as circular, but they can also include any geometric shape, including square, rectangular, triangular, oval, and any other geometric configuration. The diameter and/or width of the exit ports 30 can range from about 0.1 mm to about 10 mm.

In some embodiments, best shown in FIG. 1B, the implantable interface region 10 includes a first skin interface region 16 and a second skin interface region 18 separated therebetween by a circumferential depression 50 defining a recessed skin interface surface 38. As shown in FIG. 1B, the recessed skin interface surface 38 includes a plurality of exit ports 30. The shape of the circumferential depression 50 defined by the first and second skin interface regions 16 and 18 and the recessed skin interface surface 38 is shown as a half rectangle, wherein the under surfaces 36 and 37 are perpendicular to the recessed skin interface surface 38. In still other configurations of the implantable interface region 10, the circumferential depression 50 defined by surfaces 36, 38 and 37 may also include square, rectangular, triangular, semicircular, C-shaped or arcuate shapes. In some embodiments, the geometry of the circumferential depression can be an arcuate shaped having an opening for the skin-interface composition to exit through a controlled space. The distance between under surfaces 36 and 37 and hence the length of recessed skin interface surface 38 depicted in FIG. 1B can be adjusted to accommodate the dimensions of the exit ports 30 and can range from about 1 mm to about 20 mm. In some embodiments, the implantable interface region 10 can have at least one, at least two, at least 3 or at least four circumferential depressions 50 disposed around the periphery of the implantable interface region 10.

In some embodiments, the implantable interface region 10 can also include one or more circumferential depressions 50 wherein the exit ports 30 are circumferentially covered with a mesh material 40 to prevent entry of particles, debris, cells into the exit ports 30 and conduits 100. Mesh material 40 can be made from any biocompatible mesh material including metal, plastic or ceramic having a 5 μm to 50 μm mesh sieve opening size. The mesh material 40 can be made from metals or from one or more of Dacron®, polytetrafluorethylene, polypropylene, polyester, polyvinylidene fluoride (PVDF), silicone, Mersilene®, Marlex®, Nylon® and Teflon®.

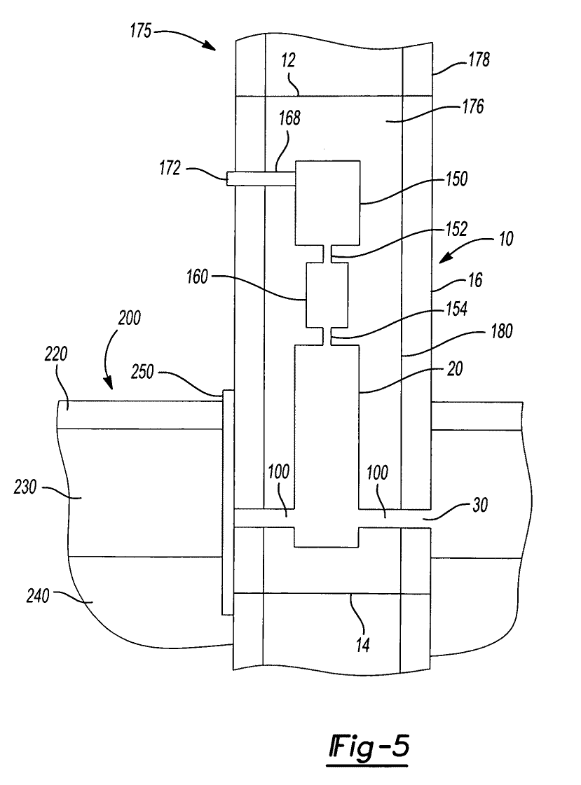

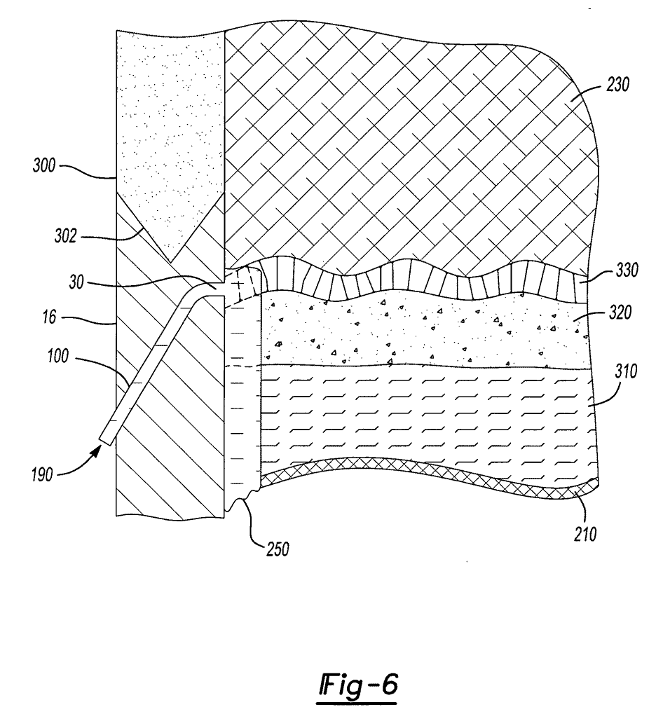

The diameter of the implantable interface region 10 can vary according to several factors, including, the diameter of the percutaneous biomedical device, the material composition of the transcutaneous medical device and/or the implantable interface region 10, the function and purpose to be served by the percutaneous biomedical device and the subject's percutaneous biomedical device insertion site. For example, FIG. 5 illustrates an implantable interface region 10 of a hollow infusion percutaneous biomedical device 175 having a hollow lumen 176. The diameter of the infusion percutaneous biomedical device 175 can range from 1 to 20 mm. Hence, the diameter of the implantable interface region 10 also ranges from 1 to 20 mm. FIGS. 6 & 7 illustrate an osseointegrated device that will support a prosthetic limb. As such, the percutaneous biomedical device is a prosthesis frame. The prosthesis frame includes an implantable interface region 10. The diameter of both the percutaneous biomedical device illustratively shown as a prosthesis frame and implantable interface region 10 can be the same or different and can range from about 0.1 mm to about 100 mm. The length and diameter of the implantable interface region 10 can be altered to meet the design requirements and functional aspects of the osseointegrated device. In some embodiments, the diameter of the implantable interface region 10 can vary from about 0.1 mm to about 100 mm. For example, as in the case of catheters and sensors which do not require load bearing function, the implantable interface region can possess diameters less than 2-5 mm providing an implantable interface region which incorporates a plurality of exit ports in fluid communication with a lumen or reservoir 190 containing a skin-interface composition.

The implantable interface region 10 can be manufactured from any biocompatible, non-toxic surgical grade plastics and metal materials including for example, ceramics, polymeric thermoplastics, silica containing synthetics, silicone containing synthetics and metals. It is preferred that the materials are surgical grade, biocompatible, non-immunogenic, non-toxic, sterilizable using chemical and/or physical sterilization (heat, ultraviolet and gamma radiation). Since these devices are to be implanted into a subject for an expended period of time, at least 2-3 weeks, the implantable interface region 10 is typically constructed from the same material used to fabricate the body of the percutaneous biomedical device. In some embodiments, the implantable interface region 10 is part of a percutaneous biomedical device including an orthopedic fixation system (for the fixation of skeletal and spinal bone structures) or an osseointegrating device, for which resiliency and load bearing capabilities are required to fixate an internal tissue such as bone or cartilage or for the support of a prosthetic device such as a prosthetic limb. In these examples, the implantable interface region 10 can be manufactured from one or more metals used for the manufacture of surgical implants, including orthopedic fixation, prosthesis frames, drug delivery needles, including surgical steel, titanium and their alloys, for example nitinol. The implantable percutaneous biomedical devices and the implantable interface region 10 can also have a coating, including a biocompatible material, such as a polymer like urethane, nylon, TPU, thermoplastic polyester elastomer, polyethyl, or silicone alone or in combination with one or more drugs, pharmaceuticals, biologics or medicaments. The coating can be applied to a percutaneous biomedical device by various methods, such as spray coating or painting.

The implantable interface region 10 also includes a lumen 20 as shown in FIGS. 1A, 1B and 4. The lumen 20 can serve as repository for the skin-interface composition that is extruded or positively released from the exit ports 30. The lumen 20 can be a reservoir for the skin-interface composition when there is no pumping mechanism needed. Alternatively, the lumen 20 receives the skin-interface composition which is actively pumped from an internal reservoir 150, or from an external reservoir 140 as shown in FIG. 4. The lumen 20 can be of any suitable dimension provided that it fits within the diameter of the implantable interface region 10. The lumen 20 is in fluid communication with the plurality of conduits 100 and the exit ports 30. The lumen 20 can also be in fluid communication and connected with an internal reservoir 150 shown illustratively in FIG. 4. The lumen 20 can be connected to a flow path to an exit port 30 connecting the implantable interface region 10 with a pump 160 and/or an external reservoir 140.

The lumen 20 can take the shape of a cylindrical void within the implantable interface region 10 as shown in FIGS. 1A and 1B, or alternatively can be of any shape and extend to any length within the percutaneous biomedical device, provided that the lumen 20 is in fluid communication with the plurality of conduits 100 and/or with the plurality of exit ports 30.

In some embodiments, the implantable interface region 10 can include a plurality of conduits 100. Conduits 100 are flow paths that are configured to channel the skin-interface composition from the internal reservoir 150 shown in FIGS. 4, 5 and 7 or from the lumen 20 to the exit ports 30. In some embodiments, one exit port 30 can be serviced by one conduit 100. The dimensions of the conduit 100 can vary according to the internal space provided by the percutaneous biomedical device and the desired flow rate of the skin-interface composition exiting from the exit ports 30. In some embodiments, the internal diameter of the conduits can be the same as the diameter of the exit ports 30, for example, ranging from about at least 0.1 mm to about at least 1 mm, to about at least 2 mm or to about at least 5 mm. The conduits can be made from any material, including, thermosetting polymeric materials, for example, known biocompatible polymers, polyethylene, polycarbonate, polyethylene terphthalate and silicone containing materials, ceramics and metals, such as surgical steel and the like.

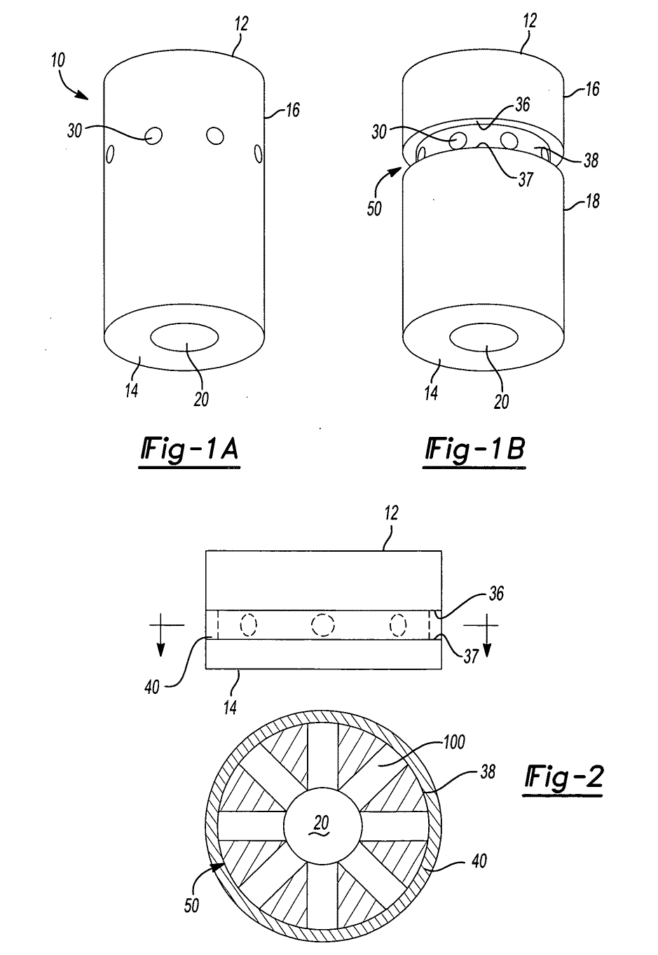

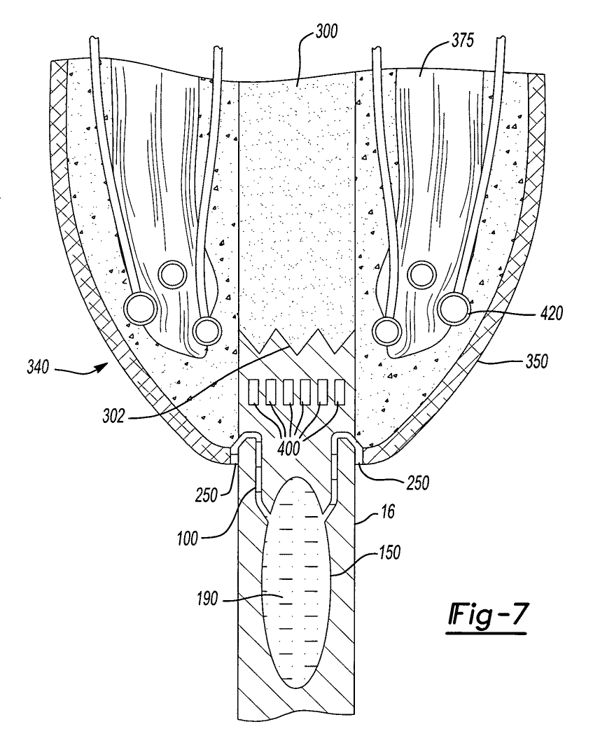

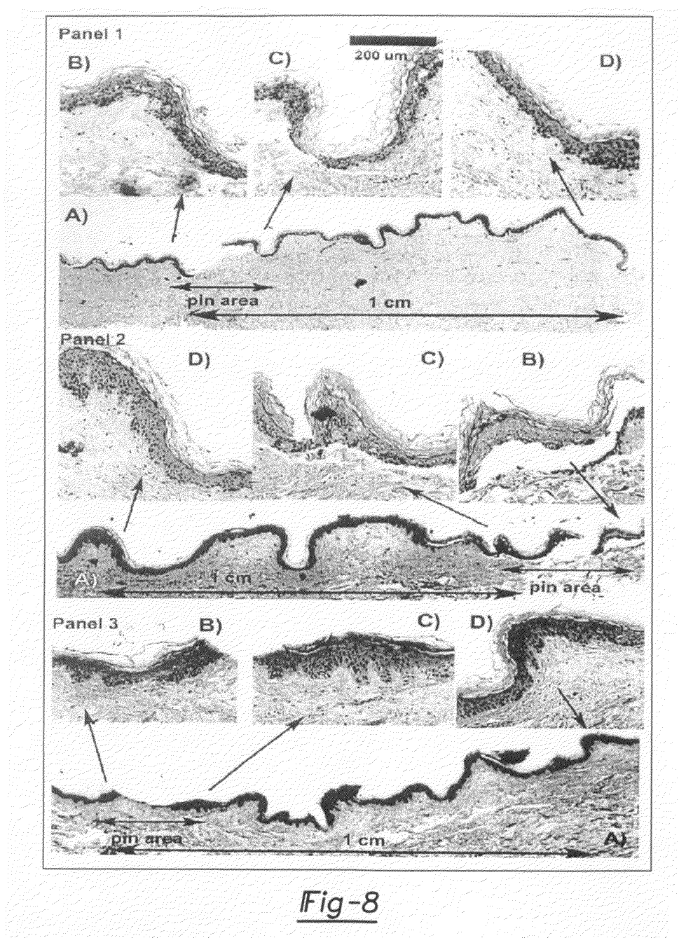

The conduits 100 can radiate from the central lumen 20 as shown in FIGS. 2 and 4. Generally as shown in these figures, the number of conduits 100 connecting the lumen 20 and exit ports 30 are variable and are usually the same as the number of exit ports 30. In some embodiments, the conduits 100 do not radiate from the lumen 20 but instead are fluid paths for skin-interface composition to travel directly from the external reservoir 140 or internal reservoir 150 as shown in FIGS. 4 and 7.

The implantable interface region 10 includes two or more exit ports 30 placed circumferentially around the first skin interface region 16 or the one or more recessed skin interface surface 38 shown in FIG. 1B. The exit port 30 is an opening of a conduit 100 that extrudes the skin-interface composition adjacent to and in contact with one or more of the various skin layers between the substratum corneum and the hypodermis. The exit ports 30 can be positioned in any arrangement around the first skin interface region 16 or the one or more recessed skin interface surface 38. However, the exit ports 30 are preferably positioned in contact and adjacent to one or more of the stratum granulosum, stratum spinosum, stratum basale, papillary region, reticular region and the hypodermis layers of the skin tissue. As merely an illustration, best represented in FIG. 5, the exit ports 30 are placed adjacent to and in contact with the reticular region of the dermis.

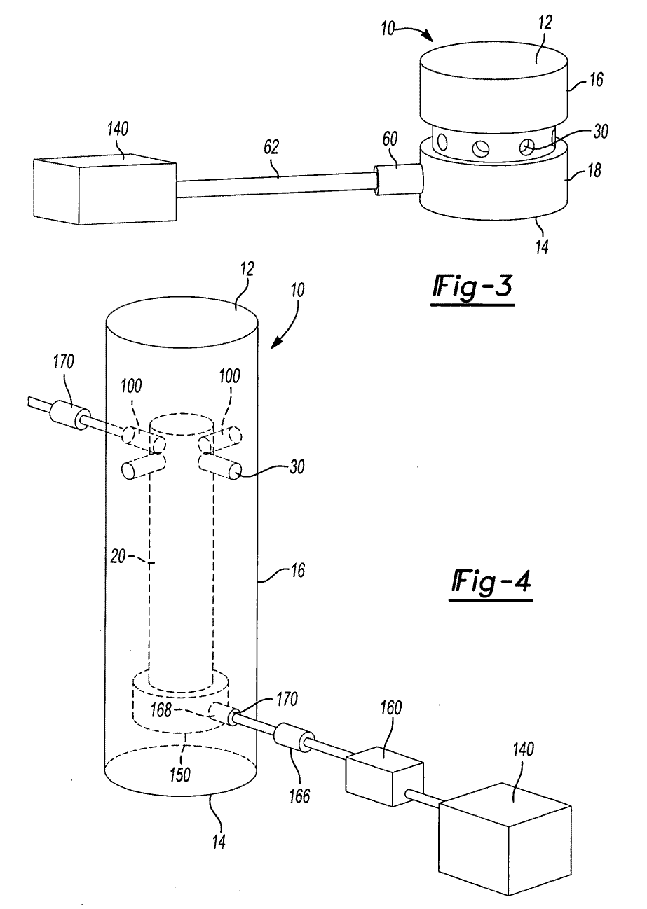

In an illustrative embodiment of the present technology, best illustrated in FIG. 4, an implantable interface region 10 as part of a thin tube such as a catheter or stent is illustratively shown having four exit ports having diameters ranging between 0.1 mm. to about 5.0 mm. In some embodiments, the diameter of the exit ports 30 can range from about at least 0.1 mm to about at least 1 mm, to about at least 2 mm, to about at least 5 mm. In some embodiments, the implantable interface region 10 can have a plurality of exit ports 30 sufficient to wet the percutaneous biomedical device by at least 50% around the periphery of the implantable interface region 10. In some embodiments, the number of exit ports 30 can include, at least about two, or at least about four, or at least about eight, or at least about ten, or at least about 12, or about 16 exit ports 30 disposed around the periphery of the implantable interface region 10. When the implantable interface region 10 includes an circumferential depression 50 as shown in FIGS. 1B and 3, the number of exit ports can range from at least about two, or at least about four, or at least about eight, or at least about ten, or at least about 12, or about 16 exit ports 30 In some embodiments, the circumferential depression 50 can be positioned subcutaneously to the stratum corneum between the stratum corneum and the hypodermis, such that the percutaneous biomedical device extrudes a skin- interface composition that will wet at least a partial portion of the implantable interface region 10 at the skin-device interface 250 as shown in FIG. 5.

In some embodiments, the implantable interface region 10 can have about at least four, or at least about eight, or at least about ten, or at least about 12, or at least about 16 exit ports disposed around the circumferential periphery of the implantable interface region.

While the skin-interface composition can be retained and provided in the lumen 20 for distribution along the conduits 100 and exiting through the exit ports 30, several embodiments can require the use of a reservoir to store an amount of skin-interface composition. To aid in the storage of the skin-interface composition, the implantable interface region 10 can be fluidly connected to one or more reservoirs. The reservoirs can be an internal reservoir 150 as exemplified in FIG. 5 and 7, or they can also be an external reservoir 140 as shown in FIGS. 3 and 4. In some embodiments, an internal reservoir 150 can hold an amount of skin-interface composition ranging from about 0.1 ul to about 25 mL in thin diameter percutaneous biomedical devices 175 such as stents, catheters, and drug delivery tubes. Externally placed reservoir 140, i.e. reservoirs that are positioned outside of the percutaneous biomedical device can store volumes of skin-interface composition ranging from 1 mL to about 200 mL.

In some embodiments, the function of the internal reservoir 150 is to temporarily store the skin-interface fluid for distribution to the conduits 100 for extrusion into the skin-device interface 250 as shown in FIG. 5. While the internal reservoir 150 size and capacity can be influenced by the size and diameter of the percutaneous biomedical device and the viscosity of the skin-interface composition, the internal reservoir 150 can be configured to be replaceable or at least changeable when used internally in the percutaneous biomedical device. This arrangement is best depicted in FIGS. 5 and 7, wherein a large percutaneous biomedical device 175 such as a prosthesis frame, can house internally a internal reservoir 150 that can hold a volume of skin-interface composition ranging from about 1 mL to about 100 mL, depending on the diameter of the percutaneous biomedical device 175. For example a 50 mL internal reservoir 150 when placed in the percutaneous biomedical device 175, can provide sufficient skin-interface composition to the skin layers adjacent to the device for at least 6-12 months when extruding 0.1 μL per min. However, when the diameter of the percutaneous biomedical device 175 is less than about 5 mm, then the skin-interface composition may need to be supplied from an external reservoir 140 i.e. a reservoir that is positioned outside of the percutaneous biomedical device 175. In some embodiments, the percutaneous biomedical device 175 shown in FIG. 5 best represents an implantable drug or nutrient delivery tube, catheter or stent. As shown in FIG. 5, an internal reservoir 150 can be connected to an outlet connector tube 172 that can permit replenishing the skin-interface composition from the outside of the percutaneous biomedical device 175. FIG. 4 is also illustrative of a percutaneous biomedical device having an external reservoir 140 that can be refilled or changed each time the level of the skin-interface composition in the external reservoir 140 is depleted or when the skin-interface composition needs to be changed to a different skin-interface composition.

In some embodiments, the external reservoir 140 of the present technology can range from macro-sized reservoirs carrying capacities ranging from 1 mL to about 100 mL are widely known in the art. Micro-reservoirs that find utility in the present percutaneous biomedical devices can include microreservoirs fabricated using conventional photolithography and self-assembly and micro electromechanical systems where microreservoirs are etched onto silicate substrates. Other larger micro-macro sized reservoirs having deformable or semi-solid linings are well known in the art of stent and drug delivery systems. The reservoirs can be made of compliant synthetic or natural materials including elastomers, polymers and polysaccharide polymers, for example, ethylene vinyl acetate, Teflon, silicone, silastic and nylon, polyvinyl alcohol, ethylene vinyl acetate, polypropylene, polycarbonate, cellulose, cellulose acetate, cellulose esters or polyether sulfone.

In some embodiments, the reservoir supplying the skin-interface composition can be implanted in the subject's body and have connectors or other conduits to supply the implantable interface region 10 with the skin-interface composition. An illustrative example of an implantable system incorporating an external pump 160 is illustrated in FIG. 4. The external reservoir 140 supplying a volume of skin-interface composition can be implanted under the skin of the subject. The implantable external reservoir 140 can have a cap or interface (not shown) to allow quick and facile endoscopic or laparoscopic filling of the reservoir by a surgeon or medically trained professional. The implantable external reservoir 140 can also be in fluid communication with a pump 160 to control the volume of skin-interface composition to be delivered to the lumen 20 or internal reservoir 150. Such implantable pumps 160 are commonly used and commercially available for drug or medicament delivery to chronically ill patients such as delivery of insulin to the liver of diabetic patients. The pump 160 can be regulated by a flow meter 166 positioned upstream from the implantable interface region 10 and/or positioned in proximate contact with the exit ports 30.

In some embodiments, the external reservoir 140 can be positioned externally to the subject and be connected to the percutaneous biomedical device 175 and implantable interface region 10 with percutaneous tubing or fluid lines. The use of external pumps has one advantage in that the reservoir can be kept completely sterile and can include large reservoirs that can be filled quickly. The external reservoir 140 can be attached to a delivery tube and a pump 160 for controlling delivery of the skin-interface composition through the delivery tube.

In some embodiments the percutaneous biomedical device 175 shown in FIGS. 4 and 5 can include a pumping mechanism 160 to transfer the contents of the external reservoir 140 or internal reservoir 150 to the lumen 20. Delivery systems can be dependent on the size of the implanted percutaneous biomedical device 175. Delivery systems can be made of a combination of one or more fluid pumps and/or micropumps, rigid or flexible, with a variety of pumping mechanisms. With reference to FIG. 4, the external pump 160 can be inserted into the patient percutaneously through an opening made in the skin or can be attached to the percutaneous biomedical device externally with a connector 168. The pump 160 can be serially connected to a flow meter 166 that can provide feedback back to the pump 160 to regulate the proper delivery of skin-interface composition to the implantable interface region 10. Control mechanisms for the delivery can be manual, mechanical or electronic. For small or thin diameter (less than 6-15 mm diameter) percutaneous biomedical devices, flexible micropumps are better suited, while for bigger percutaneous biomedical devices, externally attached fluid pumps can be advantageous. The pump 160 can be programmed to deliver specific volumes of the skin-interface composition at different pumping rates and at different times. For certain medical applications, the pump 160 and external reservoir 140 can be made of a specially designed and flexible composite microfluidic system where the materials are stored and delivered with electronically controlled mechanisms. The microfluidic system can consist of a reservoir, channels and an electronic microchip to control the extrusion of the fluid from the reservoir, for example, by using piezo-electric components.

The types of pumps 160 for use to deliver the skin-interface composition from the external reservoir 140 can be any pump system used for sub-microliter to microliter volume delivery volumes. In some embodiments, the external reservoir 140 can include peristaltic pump, diaphragm pumps, piston pumps, gradients pumps, displacement pumps such as those described in (WO/2004/001228), isocratic pumps capable of pumping nanoliter to microliter scale volumes as described in U.S. Pat. No. 7,141,161 to Ito and any pumping system that is capable of providing flow rates of the skin-interface composition through the exit ports ranging from about 0.00015 μL per min to about 10 μL per min.

In some embodiments, the external reservoir 140 and internal reservoir 150 can also include micropumps and microfluidic type devices. Micro-pumps make it possible for nanoliter quantities of liquid to be dosed accurately and flexibly. Active composites and an electronic control mechanism ensure that the low-maintenance pump works accurately. Micropumps contemplated for the present pumping mechanism can include flexible peristaltic micropumps, diaphragm micropumps, thermopneumatic peristaltic micropumps and piezo electric driven micropumps which are all useful pumping mechanisms for the percutaneous biomedical devices described herein. Micropumps and microfluidic liquid delivery systems are commercially available from thinXXS Microtechnology AG Zweibrücken Germany. Other micropumps such as the Bartels microComponents' mp5 and mp6 micropumps (Mikrotechnik GmbH Dortmund, Germany).

In some embodiments, the pump 160 can be controlled wirelessly using wireless transmitter and receivers known in the fluid delivery art.

Referring to FIG. 4, the percutaneous biomedical devices of the present technology can also include one or more flow meters 166 and 170 placed in the flow path of the skin-interface composition. Flow meters 166 and 170 which are considered useful for the present percutaneous biomedical devices can include ultrasonic flow meters capable of being coupled to micropumps commercially available from EESITEC Technologies, Skurup, SE and micro-Coriolis flow meters commercially available from ISSSYS Ypsilanti, Mich., US.

In some embodiments, pump 160 can be coupled with intelligent delivery systems e.g. sensors illustrated in FIG. 7 coupled to delivery mechanisms. The percutaneous biomedical device can also include several sensors that can detect, measure and transmit levels of inflammatory mediators, lipopolysaccharides, other microbial constituents and other indicators of interface rejection to a microprocessor that can then adjust the flow rate of the skin-interface composition being extruded from the implantable interface region 10 and/or provide diagnostic data to the illustrated in FIG. 7 indicating a need for an adjustment in the skin-interface composition, for example by indicating a need for antibiotics and anti-inflammatory agents. The information gathered by such sensors can be wirelessly transmitted to medical personnel that can adjust the skin-interface composition to include antibiotics, anti-inflammatory agents, and other medicaments to ameliorate the rejection of the device or infection occurring at the skin-device interface 250. Similarly, the medical personnel can wirelessly transmit signals to a microprocessor that controls the pump or pumps and/or reservoir(s) to release greater or lesser amounts of skin-interface composition or other actives.

The present percutaneous biomedical devices advantageously provide a skin-device interface 250 illustratively shown in FIG. 5. The extrusion of the skin-interface composition between the percutaneous biomedical device 175 and the skin of the subject provides a novel environment that can be biologically manipulated to prevent infection due to the movement of the device laterally within the skin layers. The spaces formed between the skin and the percutaneous biomedical device provides microscopic spaces for external fluids to penetrate between the skin layers and the percutaneous biomedical device. In addition, without wishing to be bound by any particular theory, it is believed that epidermal cells namely; basal cells migrate to the stratum corneum where they are shed. The present percutaneous biomedical devices provide an implantable interface region that mimics the movement of these cells by pumping fluid along at least a partial length of the first skin interface region 16 at the same rate at which the cells of the basal layer end up being shed in the stratum corneum. The layers of epidermis that regenerates have a thickness in the range of 75 to 150 microns. During a cyclic period of about 45 days, a linear displacement of about 0.002 microns per minute occurs, thus representing in terms of fluid displacement a fluid extrusion rate of 0.00015 μL/min. Hence, the present methods and devices are designed to extrude the skin-interface composition at a rate of at least 0.00015 μL/min, or at least 0.0015 μL/min, or at least 0.015 μL/min, or at least 0.15 μL/min and not more than 10.0 μL/min, or not more than 1.0 μL/min, or not more than 0.1 μL/min, or not more than 0.02 μL/min, or not more than 0.002 μL/min or not more than 0.0002 μL/min.

Other considerations in extruding a skin-interface composition from the implantable interface region and in preparing a skin-interface composition in accordance with the present technology includes preparing a composition having beneficial and regenerative activity for the epidermal cells and cells in the dermis and hypodermis. The skin-interface composition therefore includes one or more active agents that are known or believed to be beneficial to epidermal, dermal and hypodermal cells. The skin-interface composition of the present technology can also include one or more agents that are known or believed to be beneficial in the production of specific collagen species associated with healthy regenerating skin. In some embodiments, the skin-interface composition can include, as illustrative examples, one or more hydrophilic, biocompatible polymers for example, one or more polysaccharides, including glycosaminoglycans, for example dermatan sulfate, hyaluronic acid, the chondroitin sulfates, chitin, chitosan, alginate, heparin, keratan sulfate, keratosulfate, agarose, and derivatives thereof, carrageenan, guar gum, xanthan gums, locust bean gums, cellulose, polymers of cellulose, pectin and gellan. In some embodiments, the skin-interface composition can include collagen, fibronectin, laminin, and mixtures thereof. Synthetic polymers also useful in the composition can include: hydrophilic polymers including poly (vinyl alcohol), poly(ethylene glycol), poly(ethylene) oxide and mixtures thereof.

In some embodiments, the skin-interface composition can also include one or more bioactive agents including for example: natural and recombinant DNA, genes, cytokines, hormones, protein growth factors including for example: keratinocyte growth factor, epidermal growth factor, fibroblast growth factor and other known growth and differentiation factors implicated in skin regeneration and repair, pharmaceuticals, e.g., medicaments, anti-microbial agents, antibiotics, antiviral agents, microbistatic or virustatic agents, anti-inflammatory agents, such as dexamethasone and ibuprofen, anti-tumor agents, and immunomodulators; and metabolism-enhancing factors, e.g., amino acids, non-hormone peptides, ligands, vitamins, minerals, and natural extracts (e.g., botanical and marine animal extracts). The bioactive agent can also include processing, preserving, or hydration enhancing agents.

In some embodiments, a safe and effective amount of skin-interface composition is extruded from the implantable interface region 10 and coats the percutaneous biomedical device inserted through the skin along a partial length spanning a plurality of epidermal, dermal and hypodermal skin layers. The safe and effective amount of skin-interface composition creates a tissue-device interface that serves to lubricate the interface between the skin cells and the percutaneous biomedical device. Maintaining the lubricating interface also provides several advantages heretofore unexplored using such biomedical devices, for example: regeneration of skin cells along the tissue-device interface; assist in the production of healthy collagen proteins; reduce and/or prevent keratinocyte apoptosis; reduce and/or prevent infectious agents from entering into the tissue-device interfacial region and reduce and/or prevent fibrosis and scar formation at the tissue-device interface.

In some embodiments, the skin-interface composition can enhance the barrier role of such a composition placed at the tissue-skin interface by increasing the viscosity of the composition and thereby provide a cushioning effect between the device and the skin. In some embodiments, the skin-interface composition can be made to transition from a liquid to a gel by inducing a solidification transition by gellation, either by photoinitiation, exposure to water, or by native cationic or anionic species present in the tissue. The gel structure can also provide for a structural conduit for fibroblasts and other skin cells to form cellular skin networks, repopulate regions of the interface and provide a stable connection with the transcutaneous device to exclude foreign particles, microbes and other pathogenic conditions.

The skin-interface composition can be extruded from the exit ports 30 in a total amount ranging from about 1 to about 1000 μL per day, or from about 10 to about 1000, or from about 50 to about 1000 μL per day, or from about 100 to about 1000 μL per day, or from about 200 to about 1000 μL per day, or from about 1 to about 800 μL per day, or from about 1 to about 500 μL per day, or from about 1 to about 250 μL per day, or from about 1 to about 200 μL per day, or from about 1 to about 150 μL per day, or from about 1 to about 100 μL per day, or from about 1 to about 50 μL per day. In some embodiments, a safe and effective amount of skin-interface composition extruded from the exit ports 30 in total ranges from about 50 to about 500 μL per day.

### Methods Of Use

The present technology presents several novel solutions for the incompatibility of using percutaneous biomedical devices for periods of time exceeding 2-3 weeks. The present technology provides a solution to the many problems associated in maintaining a healthy skin environment for the chronic use of percutaneous biomedical devices. The normal skin condition cannot be maintained despite daily wear and tear, the prosthesis cannot be worn, no matter how accurate the integrated limb device may be. In some embodiments, the percutaneous biomedical device of the present technology can include any known surgical, dental, and cosmetic device or appliance that is inserted through the skin of a patient and affixed to an internal tissue such that the device is operable having a portion of the device situated in the skin. It is to be understood that when one end of percutaneous biomedical device has been implanted into a tissue, a device-skin junction is formed. At present, several setbacks to the success of drug-delivery, orthopedic, prosthetic, and other surgical techniques have been identified at the transcutaneous site of implantation. The percutaneous biomedical device often fails because movement of the percutaneous biomedical device at the device-skin junction results in poor adhesion between the percutaneous biomedical device and the patient's skin layers. The loss of stability at the device-skin interface has often been associated with increased infections, skin fibrosis, and inflammatory reactions resulting in implant failure.

The present technology provides for methods for connecting a variety of percutaneous biomedical devices having an implantable interface region, for example, limb prosthetics, osseointegrated devices, catheters, stents, internal metabolic sensors, pace makers, defibrillators and orthopedic implants, including fixator pins, rods and screws across the skin in a manner that renders the device stable for an extended period of time and avoids issues associated with infection, persistent inflammation or rejection. In some embodiments, the partially implanted device member can be implanted in a patient for a period of at least about 2 weeks, at least about 7 weeks and at least about 14 weeks.

The skin-interface composition can be stored in a hollow cavity or reservoir disposed within the implantable interface region as shown in FIG. 1, or alternatively, the cavity or reservoir can be attached externally to the device outside of the patient as shown in FIG. 3. In some embodiments as depicted in FIG. 3, an implantable interface region having a length of about 20 cm and a diameter of 10 cm. A central lumen is disposed within the implantable interface region in fluid communication with an external reservoir containing a skin-interface composition. The skin-interface composition can be transported from an external reservoir or fluid receptacle into the implantable interface region via a pump. The flow rate of the skin-interface composition from the reservoir to the exit ports can be controlled to provide a skin-interface composition flow rate of 1 microliter to about 1 milliliter per hour extruded from the exit port. The fluid entering and exiting through the exit port can also go through a check ball valve assembly similar to flaps or other “one way” valve mechanisms to prevent fluid, cells and other fluids from reentering the implantable interface region. In some embodiments, the implantable interface region can optionally contain an internal reservoir in fluid communication with an external reservoir and the exit ports.

The release of the skin-interface composition around the periphery of the implantable interface region is shown in FIG. 5. The skin-interface composition can be extruded from the one or more exit ports 30 and flows in proximate contact with at least a partial length of the implantable interface region 10 of the percutaneous biomedical device and the patient's skin layers, including the epidermis 220, dermis 230, and the hypodermis 240 the to form a skin-device interface 250. The skin-device interface 250 thus created enables regeneration of the skin along the skin-percutaneous biomedical device junction. By extruding the skin-interface composition comprising a biocompatible, bioactive or biostimulating composition along the device-skin interface, an intimate connection can be produced that can secure the skin to the device while simultaneously lubricating the area of contact, enabling skin cells to grow into the composition and prevent the introduction of microbes or other infectious agents into the body.

In some embodiments, the percutaneous biomedical device as illustratively shown in FIG. 5, can be implanted into the patient as normally performed. When the implantable interface region 10 and in particular the exit ports 30 has been secured or sutured into position between the stratum corneum and the hypodermis, the surgeon or physician can fill a lumen 20 or internal reservoir 150 comprising the skin-interface composition by opening the reservoir outlet connector tube 172 and inject the skin-interface composition through reservoir connector 168. Once the internal reservoir 150 has been filled with the appropriate skin-interface composition, pump 160 can commence the extrusion of the skin-interface composition by pumping the skin-interface composition from the internal reservoir 150 into the lumen 20. Once the lumen 20 has been partially or completely filled, the skin-interface composition is channeled through conduits 100 spaced around the implantable interface region. The skin-interface composition is then forced to exit the percutaneous biomedical device through the plurality of exit ports 30 and partially or completely wet at least part of the implantable interface region thereby forming an Alternatively, if the hollow cavity, fluid receptacle or reservoir containing the skin-interface composition is internal to the device, then the flow rate of the pump inside the device can be programmed to start delivery of a controlled volume per hour flow of the skin-interface composition to the exit ports of the implantable interface region. In some embodiments, the flow rate of the extruded skin-interface composition can be programmed at a prescribed rate to closely match for example, that of the normal growth rate of living skin (approximately, for example, 100 microns per 2 weeks). This rate can be translated to a flow rate of about 1 microliter to about 1000 microliters per hour. In some embodiments, the flow rate can be 100 microliters per hour or less. In some embodiments, the flow rate can be at least 10 microliters per hour.

Depending on the type of surgery undertaken and the duration the transcutaneous device must remain implanted, the surgeon or physician can monitor the physiological state of the implant, the degree of rejection and/or inflammation and/or the presence of infection at the skin-device interface and adjust the flow rate and composition of the skin-interface composition accordingly. For example, if the skin-interface composition at the skin-device interface fails to show infiltration of host skin cells into the composition, then the flow rate of the extruded skin-interface composition can be slowed to enable the growing and rejuvenated skin cells to enter the skin-interface composition and form skin networks. After a certain period, the networks in the skin-interface composition comprising collagen and other dermis layer structures are supportive to integrate the skin with the device.

The extruded skin-interface composition can contact with at least the epidermal and dermal layer of the skin where the skin cells are actively generating. As the skin cells grow, they can become intimately integrated into the skin-interface composition, which can be tailored to solidify into a solid, or semi-solid gel like material that provides a protective coating along the skin-device interface, prevent entry of infectious microbes and prevent device initiated inflammation at the skin-device interface 250.

As shown in FIG. 7, in one embodiment, a percutaneous biomedical device is shown connected to an amputated limb. The percutaneous biomedical device can include a prosthesis frame which has a first bone engagement end contacting a bone surface and a second prosthesis engagement end joined to a prosthesis limb. In some embodiments, the implantable interface region can be any length sufficient to connect a percutaneous biomedical device to a desired tissue within a subject or patient percutaneously. In some embodiments, the percutaneous biomedical device can be connected to or inserted into bone, blood vessels, particularly the interior lumen of such blood vessels, cartilage, muscle, neural tissue, such as, brain tissue, spinal cord and peripheral nerves, ligaments, and any internal organ, for example the stomach, liver, pancreas, kidneys, uterus, ovaries, testes, prostate and endocrine and paracrine glands. In some embodiments, the percutaneous device can be an implantable vascular device such as a stent, catheter, wire, lead or electrical or drug conduit.

As illustrated in FIGS. 6 and 7 the percutaneous biomedical device comprising an implantable interface region 10, and plurality of exit ports 30 can be inserted through the skin to fix or attach internal tissue shown as bone 300. The bone contacting surface of the percutaneous device forms a bone interface 302. Once the physician has connected the percutaneous biomedical device, for example a prosthesis frame or a bone fixator rod or pin, the implantable interface region 10 is adjusted such that the plurality of exit ports 30 are aligned between the dermis 230 and the stratum corneum 210. The percutaneous biomedical device is typically implanted transcutaneously for a period of at least two to three weeks. The percutaneous biomedical devices of the present technology are preferably inserted into the subject's hard or soft tissue for a period spanning at least months to several years. Once the percutaneous biomedical device has formed a bone interface 302, the extrusion of a skin-interface composition 190 along conduit 100 can commence to create an skin-device interface 250 which includes the skin-interface composition and may also include skin cells from the regions or layers of skin in contact with the implantable interface region. As merely an example of such an alignment, the exit ports 30 are aligned in FIG. 6 between the dermis and the stratum spinosum 320 and adjacent to the stratum basale 330. However, the exit ports can also be adjacent the dermis 230, the stratum spinosum 320, and the strata 310 comprising the stratum lucidum and the stratum granulosum.

In some embodiments, the bone to which a transcutaneous device can be connected, can include any bone or osseous tissue in a subject requiring fixation, support, repair, augmentation and the like. For purposes of illustration only, FIG. 7 exemplifies a bone 300 from a limb 340, for example, a femur, tibia, humerus, radius, ulna, and phalanges. In some embodiments, the bone 300 can include, the skull including cranial, facial and cochlea bones. The percutaneous biomedical device can be inserted through the skin 350 and interface with a solid or semi solid tissue such as bone 300 and form a bone interface 302. Once interfaced with bone 300, the percutaneous biomedical device can inhibit inflammation and/or infection and form a healthy skin-device interface 250 by extruding a skin-interface composition 190 from an internal reservoir 150 at a flow rate ranging between about 0.00015 μL/min to about 10 μL/min. The percutaneous biomedical device can be fitted with various wireless transmitters and receivers 400 that enable the control of the extrusion rate of the skin-interface composition exiting the exit ports (not shown). Metabolic mediators which may indicate the status of the limb or the degree of inflammation and/or infection including, muscle electrical conductivity, neural polarization and conduction, rate of lactic acid production and other physiological parameters can be sampled and communicated via implanted microfabricated neural and muscle sensors 420. As shown in FIG. 7, a limb 340 has a plurality of muscle sensors 420 implanted in the muscle 375 and can be used to determine the status of the functioning limb when connected to the percutaneous biomedical device.

In some embodiments, the percutaneous biomedical device can (having an implantable interface region 10) penetrate the skin and interface with an internal organ from the circulatory system, the auditory system, the digestive system or the nervous system. Such devices can include catheters, wires, leads sensors and other surgical or diagnostic implants that may remain partially in the patient for at least two to three weeks.

### EXAMPLES

### Example 1

### In Vitro Skin-Implant Tissue Cultures with Titanium Pin Implants

Skin Preparation and Culture Setup

Human skin was obtained from elective surgeries. Full thickness human breast skin explants from discarded material from surgeries are used. The specimens were received from healthy human subjects under University of Michigan informed consent and used immediately. After removal of subcutaneous fat, the tissue was rinsed abundantly with PBS 1X containing 125 μg/mL of gentamicin (Invitrogen/GIBCO, Carlsbad, Calif., USA) and 1.87 mg/mL of amphothericin B (Sigma-Aldrich, Milwaukee, Wis.) and placed in aliquots of the same medium for 1 hour (twice). Afterwards, the specimen was placed for another 2 hours in an incubator at 37° C. before final setup of the cultures. The culture medium used was EpiLife (Cascade Biologics, Portland, Oreg.), supplemented with EpiLife defined Growth Supplement EDGS (Cascade Biologics, Portland, Oreg.) (EDGS is an ionically balanced supplement containing purified bovine serum albumin (BSA), purified bovine transferrin, hydrocortisone, recombinant human insulin-like growth factor type-1 (rhlGF-1), prostaglandin E2 (PGE2) and recombinant human epidermal growth factor (rhEGF). In addition, the medium was supplemented with 75 μg/mL of Gentamicin and 1.125 μg/mL of amphothericin B. The final concentration of calcium used in the culture medium was 1.2 mM.

After cleaning and preparing the skin specimens, microbiological analysis of a control specimen was performed to ensure that no skin flora or other contaminants were present or remained in the explants. Microorganism analysis of the selected specimens was performed at the Microbiology Laboratory of the Burn Trauma Services at the University of Michigan Hospital. For the experiments reported, all microbiology analyses were negative. After preparation, skin specimens of approximately 1.5 cm² were cut using a scalpel and cultured for 15 days at 37° C., 5% CO₂ atmosphere in duplicate, epidermal side up at the air-liquid interface in a Transwell system consisting of 6-well Transwell carriers (Organogenesis, Canton, Mass.) and six Corning Costar supports (Fisher Scientific, Pittsburgh, Pa.). Culture media was changed every 36 hours and the stratum corneum remained constantly exposed to the air. Experiments were repeated three times, with skin from 3 different individuals (n=3). All specimens were punctured with a 3 mm diameter sterile biopsy punch (except for specific controls). Each experiment consisted of five culture plates, with four plates containing different types of control specimens.

Each culture plate had 6 wells, and each well contained one skin specimen. After cleaning, this zero day control was immediately prepared for tissue analysis as described below. The lid was designed to work with up to six reservoirs containing biological tissues and had six apertures for six percutaneous biomedical devices (six fixator pins) that formed air-tight seals to prevent contaminants from entering the reservoirs. The lids accommodated the fixator pins for use with plates PIN and PIN+BIOMAT (pin+skin-interface composition). The media for each well was changed using a small opening on the lid located next to each pin aperture, which were otherwise permanently closed. All materials were autoclaved before use in each experiment. All of the fixator pins (Stainless Steel 316, ISO 5832-9 4 mm OD and 10 cm long, (McMaster-Carr) had the same OD of 4 mm. For plates marked PIN, the pins were solid while the pins for plates PIN+BIOMAT were hollow, with a 3.6 mm ID. These hollow pins also had six orifices machined at the bottom of the pin for delivery of the biomaterial. The mixture (BIOMAT) of 0.05 wt % Dermatan Sulfate (EMD Chemicals, San Diego, Calif.) and 0.2 wt % Sodium Hyaluronate medical grade of 680 kDa. (LifeCore Biomedical, Chaska, Minn.) was prepared at room temperature and well mixed at 30° C. with a magnetic stirrer for 1 h in PBS 1X and then sterile-filtered through a 0.22 μm pore filter. A Hamilton 81320 1.0 mL syringe with a 30 gauge needle was used to deliver 100 μL of the mixture to the center of the explant puncture on specimens of plates PUNCT+BIOMAT. In specimens of plates PIN+BIOMAT the material was delivered through the top of the hollow fixator pin. The 100 μL corresponded to approximately 1.2% of the medium volume present in each well and was selected to avoid substantial increases in total volume in the culture medium. Glass tubing of 6 mm in diameter was used to cover the fixator pin aperture when the device was outside of the sterile hood to avoid bacterial contamination and was autoclaved before use in each experiment. The mixture was delivered discontinuously, at the same time that medium changes were performed, every 36 hours.

Analysis of Skin Specimens

Duplicate specimens from each plate were collected at 5 days (5D), 10 days (10D) and 15 days (15D) for analysis. Histological evaluation with haematoxylin and eosin (H&E) staining was performed for all specimens collected. For all specimens containing a perforation, tissue sections were cut from the injured areas. This type of sectioning provided the maximum viewable area of the tissue section while minimizing the distances to the pin. Blind qualitative histological analysis was performed by two independent investigators. For histological analysis, biopsies were fixed in 4% phosphate-buffered paraformaldehyde for 24 h routinely dehydrated and paraffin embedded. Serial sections were obtained at 4 μm. For light microscopy analysis, images were taken using a Nikon E800 microscope. Apoptosis and cell proliferation analyses were performed for 15D and for CNTRL+0 Day specimens in two experiments.

To analyze cell proliferation at the end of the experiment the culture medium was replaced for all specimens with fresh medium containing 400 μM/L of 5-bromo-2′-deoxyuridine (BrdU) (Sigma-Aldrich, St Louis, Mo.) and cultured for 3 hours before harvesting. Incorporated BrdU was detected by light microscopy with a horseradish peroxidase conjugated monoclonal antibody against BrdU (FrontierTM BrdU Immunohistochemistry Kit, Exalpha Biologicals, Maynard, Mass.). Four areas per slide were examined for analysis. The number of BrdU positive cells per microscopic fields was recorded and data presented as percentage of BrdU positive cells per field (mean +/−SD). Apoptotic cells were detected by labeling DNA strand breaks (TUNEL) using a commercially available kit (In SituCell Death Detection Kit, AP, Roche Diagnostics Corporation, Indianapolis, Ind.). Briefly, tissue sections were deparaffinized, rehydrated, washed in 1X PBS and labeled following manufacturer instructions, including negative and positive controls. Positive controls were prepared by incubating slides prior labeling with a solution containing 1500 U/mL of recombinant DNase I (Roche Diagnostics Corporation, Indianapolis, Ind.), 50 mM Tris-HCl, 10 mM MgCl₂ and 1 mg/mL BSA for 10 minutes. Four areas per slide were examined for analysis. The number of apoptotic cells per microscopic field was recorded and data presented as the percentage of apoptotic positive cells per field (mean +/−SD).

Results

Composite microscopy images of H&E staining of human skin interfaced with pins. Panel 1 shows microscopy images of a skin specimens implanted with a fixator pin only (no extrusion of skin-interface composition was performed). Panel 2 show microscopy images taken of a skin specimen implanted with a fixator pin where a solution of physiological saline was extruded for five days. Panel 3 shows a microscopy image of a skin specimen implanted with a fixator pin where a skin-interface composition containing a solution of skin-interface composition (hyaluronic acid and dermatan sulfate) was extruded to the skin tissues for five days. Each panel contains four pictures. At the bottom of each panel there is a 2× magnification image (labeled A) showing an extended area of the tissue. The pins were located in the areas indicated. With each panel, three 20× magnification pictures are shown, two from areas close to the location of the pins (labeled B and C) and another from an area approximately 1 cm from the pin (labeled D).

FIG. 8 depicts, a panel of images of skin tissue implanted with a percutaneous biomedical device with and without extrusion of a skin-interface composition designed to assess the effect on the tissue of a hollow pin only, pin with a control material like physiological saline and pin with a mixture of hyaluronic acid and dermatan sulfate. In addition, FIG. 8 presents a comparison of the histology of the tissues in areas surrounding the pins and areas far away from the pin. Panel 1 (top) contains images of pin specimens, panel 2 (middle) images of pins with a skin-interface composition (saline) treated specimens and panel 3 images pins with a skin-interface composition (hyaluronic acid and dermatan sulfate, (HA+DS)) Each panel contains four microscope images, with dimension and distances indicated in the FIG as a 200 micron bar. As it readily observable, all specimens maintained a very similar and fairly good epidermal architecture in the areas located far from the pin, at distances of approximately 1 cm (see magnified panel images (1.D, 2.D and 3.D). However, the areas situated closer to the location of pin show very different histology. Images of skin implant 1.B and 1.C (for pin only specimens) and images of skin implant 2.B and 2.C (for saline specimens) show a deteriorated epidermis, slightly more deteriorated in the case of the saline control. On the contrary, sections 3.B and 3.C (corresponding to a specimen treated with hyaluronic acid and dermatan sulfate) shows a healthier and less deteriorated tissue architecture.

The embodiments and the examples described herein are exemplary and not intended to be limiting in describing the full scope of the devices, compositions and methods of the present technology. Equivalent changes, modifications and variations can be made within the scope of the present technology, with substantially similar results.

What is claimed is:

1. A percutaneous biomedical device comprising a body having a lumen extending longitudinally at least partially through said body, an implantable interface region disposed along said body, said implantable interface region having a plurality of radially extending conduits through said body, each of said conduits in fluid communication with said lumen and in fluid communication with an exit port.

2. The percutaneous biomedical device of claim 1, wherein said lumen is a fluid reservoir for storing a skin-interface composition.

3. The percutaneous biomedical device of claim 1, wherein said exit port has a diameter ranging from about 0.1 mm to about 5.0 mm.

4. The percutaneous biomedical device of claim 1, wherein said exit ports are spaced circumferentially around said implantable interface region spaced equidistantly apart.

5. The percutaneous biomedical device of claim 1, wherein said exit ports are positioned on the percutaneous biomedical device such that when the percutaneous biomedical device is inserted through skin layers of a subject, said exit ports are positionable between a stratum corneum skin layer and a hypodermis skin layer of said subject.

6. The percutaneous biomedical device of claim 1 further comprising a reservoir fluidly connected to at least one of said lumen and said conduits, said reservoir operable to store a skin-interface composition.

7. The percutaneous biomedical device of claim 6, wherein said reservoir is fluidly connected to a pump operable to deliver said skin-interface composition from said reservoir to said at least one of said lumen and said conduits.

8. The percutaneous biomedical device of claim 7, wherein said pump delivers said skin-interface composition from said reservoir at a rate ranging from about 0.00015 μL/min to about 10.0 μL per min.

9. The percutaneous biomedical device of claim 1, wherein said biomedical device comprises a fixator pin, an orthopedic pin, a craniofacial pin, a rod, an orthopedic screw, an ear implant, a prosthetic limb frame, a prosthetic osseointegrated device, a catheter, a stent, a lead; a wire or a tubular device.

10. The percutaneous biomedical device of claim 9, wherein the percutaneous biomedical device comprises a fixator pin, a rod, an orthopedic screw, a prosthetic limb frame or a prosthetic osseointegrated device.

11. The percutaneous biomedical device of claim 1, wherein said implantable interface region having a first diameter and a second diameter, said second diameter being smaller than said first diameter and said second diameter defining a circumferential depression with a plurality of exit ports.

12. A percutaneous biomedical device a body having a lumen extending longitudinally at least partially through said body, an implantable interface region disposed on said body, said implantable interface region comprising a plurality of radially extending conduits extending through said body, said conduits in fluid communication with said lumen, and wherein said implantable interface region having a first diameter and a second diameter, said second diameter being smaller than said first diameter and said second diameter defining a circumferential depression with a plurality of exit ports.

13. The percutaneous biomedical device of claim 12, wherein said plurality of exit ports in said circumferential depression is covered with a mesh.

14. The percutaneous biomedical device of claim 12, said implantable interface region having a plurality of circumferential depressions each with a plurality of exit ports.

15. The percutaneous biomedical device of claim 12, further comprising a reservoir in fluid communication with at least one of said lumen and said conduits, said reservoir operable to store a skin-interface composition, a pump in fluid communication with said reservoir and said lumen configured to deliver said skin interface composition to at least one of said plurality of exit ports, wherein said plurality of exit ports are positioned on the percutaneous biomedical device such that when the percutaneous biomedical device is inserted through skin layers of a subject, said exit ports are positionable between a stratum corneum skin layer and a hypodermis skin layer of said subject.

16. A method for implanting a percutaneous biomedical device comprising:
a. providing a percutaneous biomedical device comprising an implantable interface region and a lumen configured for receiving a fluid comprising a skin-interface composition, said implantable interface region comprising a plurality of radially extending conduits, said conduits in fluid communication with said lumen and with a plurality of exit ports;
b. implanting said percutaneous biomedical device in a tissue;
c. aligning said plurality of exit ports adjacent to a skin layer; and
d. extruding said skin-interface composition through said exit ports to contact said skin layer with said skin-interface composition.

17. The method for implanting a percutaneous biomedical device of claim 16, wherein aligning said plurality of exit ports adjacent to a skin layer comprises aligning said percutaneous biomedical device such that said plurality of exit ports are disposed adjacent skin layers selected from the group consisting of stratum corneum, stratum lucidum, stratum granulosum, stratum spinosum, stratum basale, dermis, hypodermis and combinations thereof.

18. The method for implanting a percutaneous biomedical device of claim 16, wherein providing a percutaneous biomedical device further comprises providing a percutaneous biomedical device comprising an implantable interface region, and a lumen for receiving a fluid, said implantable interface region comprising a plurality of radially extending conduits in fluid communication with said lumen, and wherein said implantable interface region having a first diameter and a second diameter, said second diameter being smaller than said first diameter and said second diameter defining a circumferential depression with a plurality of exit ports.

19. The method for implanting a percutaneous biomedical device of claim 16, wherein implanting said percutaneous biomedical device in a tissue comprises implanting said percutaneous biomedical device in contact with a bone tissue.

20. The method for implanting a percutaneous biomedical device of claim 16, wherein implanting said percutaneous biomedical device in a tissue comprises implanting said percutaneous biomedical device in contact with a soft tissue selected from the group consisting of a vascular lumen, brain tissue, spinal cord, muscle, an organ and connective tissues in connection thereof.
